(19) United States
(12) Patent Application Publication
(10) Pub. No.: US 2021/0259769 A1
(43) Pub. Date: Aug. 26, 2021

(54) OPTICAL BALLOON CATHETERS AND METHODS FOR MAPPING AND ABLATION

(71) Applicant: St. Jude Medical, Cardiology Division, Inc., St. Paul, MN (US)

(72) Inventors: Chunlan JIANG, Northridge, CA (US); Louis-Philippe Richer, Montreal, Quebec (CA)

(21) Appl. No.: 16/973,185

(22) PCT Filed: Aug. 14, 2019
(86) PCT No.: PCT/US2019/046483

§ 371 Date: Dec. 8, 2020

Related U.S. Application Data
(60) Provisional application No. 62/765,170, filed on Aug. 17, 2018.

(51) Int. Cl. A61B 18/20; A61B 5/00

(52) U.S. Cl. CPC A61B 18/20; A61B 2018/0022; A61B 5/6853; A61B 5/0084

(57) ABSTRACT

Systems and methods for optical balloon catheters are provided. A catheter includes a distal section including an optically transparent balloon, a first optical array positioned within the balloon, wherein the first optical array is configured to at least one of ablate tissue and sense at least one tissue property, and a second optical array positioned outside the balloon, wherein the second optical array is configured to at least one of ablate tissue and sense at least one tissue property.

### CROSS REFERENCE TO RELATED APPLICATIONS

This application claims priority to provisional application Ser. No. 62/765,170, filed Aug. 17, 2018, which is incorporated herein in its entirety.

### FIELD OF THE DISCLOSURE

This disclosure relates to mapping and ablating tissue, and more particularly, this disclosure relates to optical balloon catheters for mapping and ablation.

### BACKGROUND

It is known that various computer-based systems and computer-implemented methodologies can be used to generate multi-dimensional surface models of geometric structures, such as, for example, anatomic structures. More specifically, a variety of systems and methods have been used to generate multi-dimensional surface models of the heart and/or particular portions thereof.

The human heart muscle routinely experiences electrical currents traversing its many surfaces and ventricles, including the endocardial surfaces. Just prior to each heart contraction, the heart muscle is said to “depolarize” and “repolarize,” as electrical currents spread across the heart and throughout the body. In healthy hearts, the surfaces and ventricles of the heart will experience an orderly progression of a depolarization wave. In unhealthy hearts, such as those experiencing atrial arrhythmia, including for example, ectopic atrial tachycardia, atrial fibrillation, and atrial flutter, the progression of the depolarization wave may not be so orderly. Arrhythmias may persist as a result of scar tissue or other obstacles to rapid and uniform depolarization. These obstacles may cause depolarization waves to repeat a circuit around some part of the heart. Atrial arrhythmia can create a variety of dangerous conditions, including irregular heart rates, loss of synchronous atrioventricular contractions, and stasis of blood flow, all of which can lead to a variety of ailments and even death.

Medical devices, such as, for example, electrophysiology (EP) catheters, are used in a variety of diagnostic and/or therapeutic medical procedures to correct such heart arrhythmias. Typically in a procedure, a catheter is manipulated through a patient's vasculature to a patient's heart, for example, and carries one or more electrodes that may be used for mapping, ablation, diagnosis, and/or to perform other functions. Once at an intended site, treatment may include radio frequency (RF) ablation, cryoablation, lasers, chemicals, high-intensity focused ultrasound, etc. An ablation catheter imparts such ablative energy to cardiac tissue to create a lesion in the cardiac tissue. This lesion disrupts undesirable electrical pathways and thereby limits or prevents stray electrical signals that lead to arrhythmias. As readily apparent, such treatment requires precise control of the catheter during manipulation to, from, and at the treatment site, which can invariably be a function of a user's skill level.

For complex arrhythmia ablation procedures, three-dimensional analysis of cardiac tissue is utilized. As technology has advanced, tools for adequate mapping and substrate identification have also evolved, providing physicians with a better understanding of the origin of arrhythmias, as well as their progression and diseased state. For example intramural scar tissue may facilitate intramural or transmural reentry circuits, which may be detected by prolonged transmural activation intervals.

Tools leveraging optical principles are emerging in the EP therapeutic area. For example, at least some known ablation systems are optical (e.g., laser) ablation systems. Optical tools are capable of delivering high precision, relatively quick therapy. However, optical technology is still far from being fully leveraged in cardiac EP.

Further, arrhythmogenic substrate characterization is current based on electrical recordings primarily. However, molecular imaging has recently provided new insights into arrhythmogenic substrate characterization. Unfortunately, at least some known molecular imaging procedures are relatively length and complex.

### BRIEF SUMMARY OF THE DISCLOSURE

In one embodiment, the present disclosure is directed to a catheter. The catheter includes a distal section including an optically transparent balloon, a first optical array positioned within the balloon, wherein the first optical array is configured to at least one of ablate tissue and sense at least one tissue property, and a second optical array positioned outside the balloon, wherein the second optical array is configured to at least one of ablate tissue and sense at least one tissue property.

In another embodiment, the present disclosure is directed to a catheter. The catheter includes a distal section including an optically transparent balloon, an optical array positioned within the balloon, wherein the optical array is configured to at least one of ablate tissue and sense at least one tissue property, and an electrode array comprising a plurality of electrodes, wherein the electrode array is configured to sense at least one tissue property.

In yet another embodiment, the present disclosure is directed to a method of using a catheter. The method includes deploying the catheter to a target tissue location, the catheter including a distal section having optically transparent balloon, a first optical array positioned within the balloon, and at least one of i) a second optical array positioned outside the balloon, and ii) an electrode array comprising a plurality of electrodes. The method further includes using at least the first optical array, at least one of i) sensing at least one property of the target tissue, and ii) ablating the target tissue.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic and diagrammatic view of a system for performing at least one of a diagnostic and a therapeutic medical procedure in accordance with present teachings.

FIG. 2 is a schematic and diagrammatic view of one embodiment of a visualization, navigation, and mapping subsystem that may be used with the system shown in FIG. 1.

FIG. 3 is a schematic side view of one exemplary embodiment of an optical balloon catheter that may be used with the system shown in FIG. 1.

FIG. 4 is a schematic side view of a distal section that may be used with the catheter shown in FIG. 3.

FIG. 5 is a schematic end view of the distal section shown in FIG. 4.

FIG. 6 is a schematic side view of an alternative balloon catheter that may be used with the system shown in FIG. 1.

FIG. 7 illustrates using an optical array of the catheter shown in FIG. 6 to map tissue.

FIG. 8 illustrates using an electrode array of the catheter shown in FIG. 6 to map tissue.

FIG. 9 illustrates a distal section of the catheter shown in FIG. 6 in a collapsed configuration.

FIGS. 10 and 11 illustrate a distal section of the catheter shown in FIG. 6 in an expanded configuration.

Corresponding reference characters indicate corresponding parts throughout the several views of the drawings.

### DETAILED DESCRIPTION OF THE DISCLOSURE

The disclosure provides systems and methods for optical balloon catheters. A catheter includes a distal section including an optically transparent balloon, a first optical array positioned within the balloon, wherein the first optical array is configured to at least one of ablate tissue and sense at least one tissue property, and a second optical array positioned outside the balloon, wherein the second optical array is configured to at least one of ablate tissue and sense at least one tissue property.

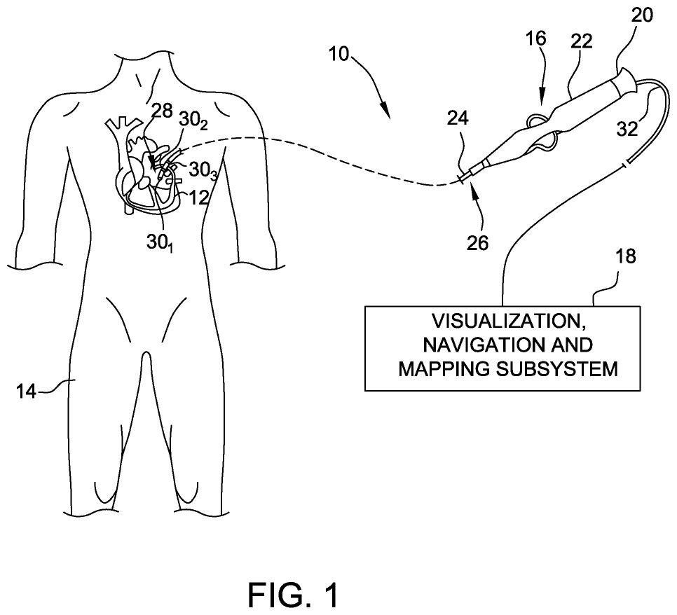

Referring now to the drawings wherein like reference numerals are used to identify identical components in the various views, FIG. 1 illustrates one exemplary embodiment of a system 10 for performing one or more diagnostic and/or therapeutic functions on or for a tissue 12 of a body 14. In an exemplary embodiment, tissue 12 includes heart or cardiac tissue within a human body 14. It should be understood, however, that system 10 may find application in connection with a variety of other tissues within human and non-human bodies, and therefore, the present disclosure is not meant to be limited to the use of system 10 in connection with only cardiac tissue and/or human bodies.

System 10 may include a medical device (e.g., a catheter 16) and a subsystem 18 for the visualization, navigation, and/or mapping of internal body structures (hereinafter referred to as the “visualization, navigation, and mapping subsystem 18”, “subsystem 18”, or “mapping system”).

In this embodiment, medical device includes a catheter 16, such as, for example, an electrophysiology catheter. In other exemplary embodiments, medical device may take a form other than catheter 16, such as, for example and without limitation, a sheath or catheter-introducer, or a catheter other than an electrophysiology catheter. For clarity and illustrative purposes only, the description below will be limited to embodiments of system 10 wherein medical device is a catheter (catheter 16).

Catheter 16 is provided for examination, diagnosis, and/or treatment of internal body tissues such as tissue 12. Catheter 16 may include a cable connector 20 or interface, a handle 22, a shaft 24 having a proximal end 26 and a distal end 28 (as used herein, “proximal” refers to a direction toward the end of catheter 16 near handle 22, and “distal” refers to a direction away from handle 22), and one or more electrophysiological (EP) sensors, such as, for example and without limitation, a plurality of electrodes 30 (i.e., 30ₖ, 30₂, . . . , 30_{N}), mounted in or on shaft 24 of catheter 16 at or near distal end 28 of shaft 24. The EP sensors may include, for example, electrode sensors and/or optical sensors, as described in detail herein.

In this embodiment, the EP sensors are configured to both acquire EP data corresponding to tissue 12, and to produce signals indicative of its three-dimensional (3-D) position (hereinafter referred to as “positioning data”). In another embodiment, catheter 16 may include one or more positioning sensors. In one such embodiment, EP sensors are configured to acquire EP data relating to tissue 12, while the positioning sensor(s) is configured to generate positioning data indicative of the 3-D position thereof, which may be used to determine the 3-D position of each EP sensor. In other embodiments, catheter 16 may further include other conventional components such as, for example and without limitation, steering wires and actuators, irrigation lumens and ports, pressure sensors, contact sensors, temperature sensors, additional electrodes and corresponding conductors or leads, and/or ablation elements (e.g., ablation electrodes, high intensity focused ultrasound ablation elements, and the like).

Connector 20 provides mechanical and electrical connection(s) for one or more cables 32 extending, for example, from visualization, navigation, and mapping subsystem 18 to one or more EP sensors or the positioning sensor(s) mounted on catheter 16. In other embodiments, connector 20 may also provide mechanical, electrical, and/or fluid connections for cables extending from other components in system 10, such as, for example, an ablation system and a fluid source (when catheter 16 includes an irrigated catheter). Connector 20 is disposed at proximal end 26 of catheter 16.

Handle 22 provides a location for a user to hold catheter 16 and may further provide means for steering or guiding shaft 24 within body 14. For example, handle 22 may include means to manipulate one or more steering wires extending through catheter 16 to distal end 28 of shaft 24 to steer shaft 24. It will be appreciated by those of skill in the art that the construction of handle 22 may vary. In other embodiments, the control of catheter 16 may be automated such as by being robotically driven or controlled, or driven and controlled by a magnetic-based guidance system. Accordingly, catheters controlled either manually or automatically are both within the spirit and scope of the present disclosure.

Shaft 24 is an elongate, tubular, and flexible member configured for movement within body 14. Shaft 24 supports, for example and without limitation, electrodes 30, other EP sensors or positioning sensors mounted thereon, associated conductors, and possibly additional electronics used for signal processing or conditioning. Shaft 24 may also permit transport, delivery and/or removal of fluids (including irrigation fluids, cryogenic ablation fluids, and body fluids), medicines, and/or surgical tools or instruments. Shaft 24, which may be made from conventional materials such as polyurethane, defines one or more lumens configured to house and/or transport electrical conductors, fluids, or surgical tools. Shaft 24 may be introduced into a blood vessel or other structure within body 14 through a conventional introducer. Shaft 24 may then be steered or guided through body 14 to a desired location such as tissue 12. Distal end 28 of shaft 24 may be the main portion of catheter 16 that contains electrodes 30 or other sensors for acquiring EP data and positioning data.

Visualization, navigation, and mapping subsystem 18 may determine the positions of electrodes 30 or other EP sensors. These positions may be projected onto a geometrical anatomical model. In some embodiments, visualization, navigation, and mapping subsystem 18 includes a magnetic field-based system. For example visualization, navigation, and mapping subsystem 18 may include an electrical field- and magnetic field-based system such as the ENSITE PRECISION™ system commercially available from Abbott Laboratories, and generally shown with reference to U.S. Pat. No. 7,263,397 entitled “Method and Apparatus for Catheter Navigation and Location and Mapping in the Heart”, the entire disclosure of which is incorporated herein by reference. In such embodiments, distal end 28 may include at least one magnetic field sensor—e.g., magnetic coils (not shown). If two or more magnetic field sensors are utilized, a full six-degree-of-freedom registration of magnetic and spatial coordinates could be accomplished without having to determine orthogonal coordinates by solving for a registration transformation from a variety of positions and orientations. Further benefits of such a configuration may include advanced dislodgement detection and deriving dynamic field scaling since they may be self-contained.

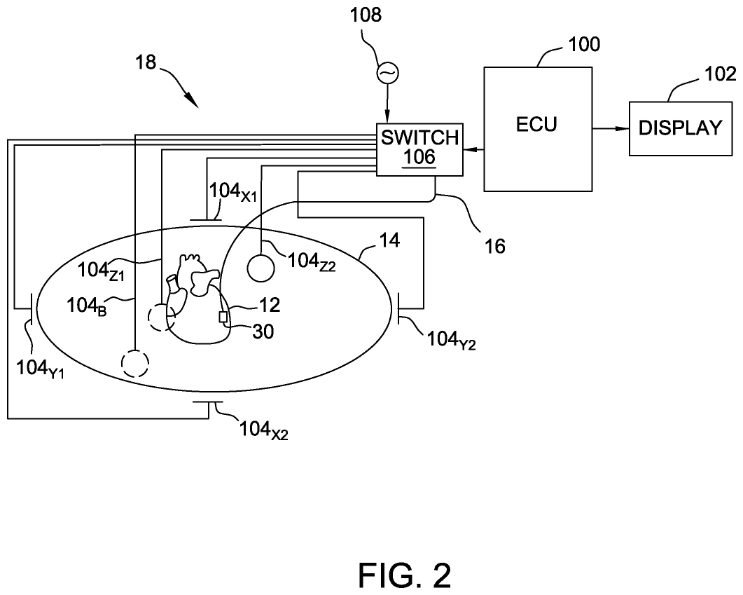

With reference to FIGS. 1 and 2, the visualization, navigation, and mapping subsystem 18 will now be described. The visualization, navigation, and mapping subsystem 18 is provided for visualization, navigation, and/or mapping of internal body structures and/or medical devices. In an exemplary embodiment, the subsystem 18 may contribute to the functionality of the system 10 in two principal ways. First, the subsystem 18 may provide the system 10 with a geometrical anatomical model representing at least a portion of the tissue 12. Second, the subsystem 18 may provide a means by which the position coordinates (x, y, z) of the electrodes 30 (or generally, EP sensors) may be determined as they measure EP data for analyses performed as part of the system 10. In certain embodiments, positioning sensors (e.g., electrical-field based or magnetic-field based) that are fixed relative to the EP sensors are used to determine the position coordinates. The positioning sensors provide the subsystem 18 with positioning data sufficient to determine the position coordinates of the EP sensors. In other embodiments, position coordinates may be determined from the EP sensors themselves by using, for example, voltages measured by the EP sensors.

Visualization, navigation, and mapping subsystem 18 may utilize, for example, the ENSITE NAVX™ system commercially available from Abbott Laboratories, and as generally shown with reference to U.S. Pat. No. 7,263,397 titled “Method and Apparatus for Catheter Navigation and Location and Mapping in the Heart,” the entire disclosure of which is incorporated herein by reference, or the ENSITE™ VELOCITY™ or ENSITE PRECISION™ system running a version of the NAVX™ software.

In other exemplary embodiments, subsystem 18 may utilize systems other than electric field-based systems. For example, subsystem 18 may comprise a magnetic field-based system such as the CARTO™ system commercially available from Biosense Webster, and as generally shown with reference to one or more of U.S. Pat. No. 6,498,944 entitled “Intrabody Measurement”; U.S. Pat. No. 6,788,967 entitled “Medical Diagnosis, Treatment and Imaging Systems”; and U.S. Pat. No. 6,690,963 entitled “System and Method for Determining the Location and Orientation of an Invasive Medical Instrument,” the disclosures of which are incorporated herein by reference in their entireties.

In yet another exemplary embodiment, subsystem 18 may include a magnetic field-based system such as the GMPS system commercially available from MediGuide Ltd., and as generally shown with reference to one or more of U.S. Pat. No. 6,233,476 entitled “Medical Positioning System”; U.S. Pat. No. 7,197,354 entitled “System for Determining the Position and Orientation of a Catheter”; and U.S. Pat. No. 7,386,339 entitled “Medical Imaging and Navigation System,” the disclosures of which are incorporated herein by reference in their entireties.

In a further exemplary embodiment, subsystem 18 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety. In yet still other exemplary embodiments, the subsystem 18 may comprise or be used in conjunction with other commonly available systems, such as, for example and without limitation, fluoroscopic, computed tomography (CT), and magnetic resonance imaging (MRI)-based systems.

In one embodiment wherein subsystem 18 includes an electric field-based system, and as described above, catheter 16 includes a plurality of electrodes 30 configured to both acquire EP data and produce signals indicative of catheter position and/or orientation information (positioning data). Subsystem 18 may use, for example and without limitation, time-division multiplexing or other similar techniques such that positioning data indicative of the position of electrodes 30 is measured intermittently with EP data. Thus, an electric field used to locate electrodes 30 may be activated between measurements of EP data, and electrodes 30 may be configured to measure both EP data and the electric field from subsystem 18, though at different times.

In other embodiments, however, wherein electrodes 30 may not be configured to produce positioning data, catheter 16 may include one or more positioning sensors in addition to electrodes 30. In one such embodiment, catheter 16 may include one or more positioning electrodes configured to generate signals indicative of the 3-D position or location of the positioning electrode(s). Using the position of the positioning electrode(s) along with a known configuration of catheter 16 (e.g., the known spacing between the positioning electrode(s) and electrodes 30) the position or location of each electrode 30 can be determined.

Alternatively, in another embodiment, rather than including an electric-field based system, subsystem 18 includes a magnetic field-based system. In such an embodiment, catheter 16 may include one or more magnetic sensors (e.g., coils) configured to detect one or more characteristics of a low-strength magnetic field. The detected characteristics may be used, for example, to determine a 3-D position or location for the magnetic sensors(s), which may then be used with a known configuration of the catheter 16 to determine a position or location for each electrode 30.

For purposes of clarity and illustration only, subsystem 18 will be described hereafter as comprising an electric field-based system, such as, for example, the ENSITE™ VELOCITY™ system identified above. Further, the description below will be limited to an embodiment of system 10 wherein electrodes 30 are configured to both acquire EP data and produce positioning data. It will be appreciated in view of the above, however, that the present disclosure is not meant to be limited to an embodiment wherein subsystem 18 includes an electric field-based system or electrodes 30 serve a dual purpose or function. Accordingly, embodiments wherein subsystem 18 is other than an electric field-based system, and catheter 16 includes positioning sensors in addition to electrodes 30 remain within the spirit and scope of the present disclosure.

With reference to FIGS. 1 and 2, in this embodiment subsystem 18 may include an electronic control unit (ECU) 100 and a display device 102. Alternatively, one or both of ECU 100 and display device 102 may be separate and distinct from, but electrically connected to and configured for communication with, subsystem 18. Subsystem 18 may still further include a plurality of patch electrodes 104, among other components. With the exception of a patch electrode 104_{B} called a “belly patch,” patch electrodes 104 are provided to generate electrical signals used, for example, in determining the position and orientation of catheter 16, and in the guidance thereof. Catheter 16 may be coupled to ECU 100 or subsystem 18 with a wired or wireless connection.

In one embodiment, patch electrodes 104 are placed orthogonally on the surface of body 14 and are used to create axes-specific electric fields within body 14. For instance, patch electrodes 104_{X1}, 104_{X2} may be placed along a first (x) axis. Patch electrodes 104_{Y1}, 104_{Y2} may be placed along a second (y) axis, and patch electrodes 104_{Z1}, 104_{Z2} may be placed along a third (z) axis. These patches may act as a pair or dipole. In addition or in the alternative, the patches may be paired off an axis or paired in series, e.g., 104_{X1} is paired with 104_{Y1}, then 104_{X2}, 104_{Z1}, 104_{Z2}. In addition, multiple patches may be placed on one axis, e.g., under the patient. Each of the patch electrodes 104 may be coupled to a multiplex switch 106. In this embodiment, ECU 100 is configured, through appropriate software, to provide control signals to switch 106 to thereby sequentially couple pairs of electrodes 104 to a signal generator 108. Excitation of each pair of electrodes 104 generates an electric field within body 14 and within an area of interest such as tissue 12. Voltage levels at the non-excited electrodes 104, which are referenced to the belly patch 104_{B}, are filtered and converted and provided to ECU 100 for use as reference values.

With electrodes 30 electrically coupled to ECU 100, electrodes 30 are placed within electrical fields that patch electrodes 104 create in body 14 (e.g., within the heart) when patch electrodes 104 are excited. Electrodes 30 experience voltages that are dependent on the respective locations between patch electrodes 104 and the respective positions of electrodes 30 relative to tissue 12. Voltage measurement comparisons made between electrodes 30 and patch electrodes 104 can be used to determine the position of each electrode 30 relative to tissue 12. Accordingly, ECU 100 is configured to determine position coordinates (x, y, z) of each electrode 30. Further, movement of electrodes 30 near or against tissue 12 (e.g., within a heart chamber) produces information regarding the geometry of tissue 12.

The information relating to the geometry of the tissue 12 may be used, for example, to generate models and/or maps of anatomical structures that may be displayed on a display device, such as, for example, display device 102. Information received from electrodes 30 can also be used to display on display device 102 the location and orientation of the electrodes 30 and/or the tip of catheter 16 relative to tissue 12. Accordingly, among other things, ECU 100 may provide a means for generating display signals for display device 102 and for creating a graphical user interface (GUI) on display device 102. It should be noted that in some instances where the present disclosure refers to objects as being displayed on the GUI or display device 102, this may actually mean that representations of these objects are being displayed on GUI or the display device 102.

It should also be noted that while in an exemplary embodiment ECU 100 is configured to perform some or all of the functionality described above and below, in another exemplary embodiment, ECU 100 may be separate and distinct from subsystem 18, and subsystem 18 may have another ECU configured to perform some or all of the functionality described herein. In such an embodiment, that ECU could be electrically coupled to, and configured for communication with, ECU 100. However, for purposes of clarity and illustration only, the description below will be limited to an embodiment wherein ECU 100 is shared between subsystem 18 and system 10 and is configured to perform the functionality described herein. Still further, despite reference to a “unit,” ECU 100 may include a number or even a considerable number of components (e.g., multiple units, multiple computers, etc.) for achieving the exemplary functions described herein. In some embodiments, then, the present disclosure contemplates ECU 100 as encompassing components that are in different locations.

ECU 100 may include, for example, a programmable microprocessor or microcontroller, or may comprise an application specific integrated circuit (ASIC). ECU 100 may include a central processing unit (CPU) and an input/output (I/O) interface through which ECU 100 may receive a plurality of input signals including, for example, signals generated by patch electrodes 104 and positioning sensors. ECU 100 may also generate a plurality of output signals including, for example, those used to control display device 102 and switch 106. ECU 100 may be configured to perform various functions, such as those described in greater detail above and below, with appropriate programming instructions or code. Accordingly, in one embodiment, ECU 100 is programmed with one or more computer programs encoded on a computer-readable storage medium for performing the functionality described herein.

In addition to the above, ECU 100 may further provide a means for controlling various components of system 10 including, but not limited to, switch 106. In operation, ECU 100 generates signals to control switch 106 to thereby selectively energize patch electrodes 104. ECU 100 receives positioning data from catheter 16 reflecting changes in voltage levels and from the non-energized patch electrodes 104. ECU 100 uses the raw positioning data produced by patch electrodes 104 and electrodes 30, and corrects the data to account for respiration, cardiac activity, and other artifacts using known or hereinafter developed techniques. The corrected data, which comprises position coordinates corresponding to each of electrodes 30 (e.g., (x, y, z)), may then be used by ECU 100 in a number of ways, such as, for example and without limitation, to create a geometrical anatomical model of an anatomical structure or to create a representation of catheter 16 that may be superimposed on a map, model, or image of tissue 12 generated or acquired by ECU 100.

ECU 100 may be configured to construct a geometrical anatomical model of tissue 12 for display on display device 102. ECU 100 may also be configured to generate a GUI through which a user may, among other things, view a geometrical anatomical model. ECU 100 may use positioning data acquired from electrodes 30 or other EP sensors on distal end 28 or from another catheter to construct the geometrical anatomical model. In one embodiment, positioning data in the form of a collection of data points may be acquired from surfaces of tissue 12 by sweeping distal end 28 of catheter 16 along the surfaces of tissue 12. From this collection of data points, ECU 100 may construct the geometrical anatomical model. One way of constructing the geometrical anatomical model is described in U.S. patent application Ser. No. 12/347,216 entitled “Multiple Shell Construction to Emulate Chamber Contraction with a Mapping System,” the entire disclosure of which is incorporated herein by reference. Moreover, the anatomical model may comprise a 3-D model or a two-dimensional (2-D) model. As will be described in greater detail below, a variety of information may be displayed on the display device 102, and in the GUI displayed thereon, in particular, in conjunction with the geometrical anatomical model, such as, for example, EP data, images of catheter 16 and/or electrodes 30, metric values based on EP data, HD surface maps, and HD composite surface maps.

To display the data and images that are produced by ECU 100, display device 102 may include one or more conventional computer monitors other display devices well known in the art. It is desirable for display device 102 to use hardware that avoids aliasing. To avoid aliasing, the rate at which display device 102 is refreshed should be at least as fast as the frequency with which ECU 100 is able to continuously compute various visual aids, such as, for example, HD surface maps.

As described above, the plurality of electrodes 30 or other EP sensors disposed at distal end 28 of catheter 16 are configured to acquire EP data. The data collected by the EP sensors may be collected simultaneously. In one embodiment, EP data may include at least one electrogram. An electrogram indicates the voltage measured at a location (e.g., a point along tissue 12) over a period of time. By placing a high density of electrodes 30 or other EP sensors on distal end 28, ECU 100 may acquire a set of electrograms measured from adjacent locations in tissue 12 during the same time period. The adjacent electrode 30 locations on distal end 28 may collectively be referred to as a “region.”

ECU 100 may also acquire times at which electrograms are measured, the positions from which electrograms are measured, and the distances between electrodes 30 or other EP sensors. As for timing data, ECU 100 may track, maintain, or associate timing data with the voltages of each electrode 30 or other EP sensor as measured. In addition, the 3-D position coordinates of each electrode 30 or other EP sensor as it acquires data may be determined, for example, as described above by visualization, navigation, and mapping subsystem 18. ECU 100 may be configured to continuously acquire position coordinates of electrodes 30 or other EP sensors, especially when electrodes 30 or other EP sensors are measuring EP data. Because ECU 100 may know the spatial distribution of electrodes 30 or other EP sensor of each distal end 28 configuration (e.g., matrix-like, spiral, basket, etc.), ECU 100 may recognize from the position coordinates of electrodes 30 or other EP sensors which configuration of distal end 28 is deployed within a patient. Furthermore, the distances between electrodes 30 or other EP sensors may be known by ECU 100 because electrodes 30 or other EP sensors may be precisely and strategically arranged in a known spatial configuration. Thus, if distal end 28 is not deformed, a variety of analyses may use the known distances between electrodes 30 or other EP sensors without having to obtain the coordinate positions from the subsystem 18 to solve for the distances between electrodes 30 or other EP sensors.

With ECU 100 having voltage, timing, and position data corresponding to respective electrodes 30 or other EP sensors in addition to the known spatial configuration of electrodes 30 or other EP sensors, many comparative temporal and spatial analyses may be performed, as described below. Some of these analyses lead to creation of HD surface maps representing activation patterns from tissue 12, which are possible in part because of the high density of electrodes 30 or other EP sensors at distal end 28 of shaft 24. By providing a high density of electrodes 30 or other EP sensors at distal end 28, the accuracy and resolution of HD surface maps produced by system 10 are enhanced.

With respect to capturing or collecting EP data measured by the high density of electrodes 30 or other EP sensors, in one embodiment, ECU 100 may be programmed to continuously record and analyze data in real-time or near real-time. In another embodiment, a user may specify through a user input device a time window (e.g., 200 ms, 30 seconds, 10 minutes etc.) during which ECU 100 may capture data measured from electrodes 30 or other EP sensors. The user input device may include, for example and without limitation, a mouse, a keyboard, a touch screen, and/or the like. It should be noted that in one embodiment, electrodes 30 may continuously measure voltages along tissue 12, and ECU 100 may selectively capture or record such voltages from electrodes 30. In still another embodiment, electrodes 30 measure voltages in accordance with a sampling rate or command from ECU 100. Once distal end 28 of shaft 24 is positioned near or along tissue 12 as desired, the user could prompt a trigger for the time window. The user may configure the trigger for the time window to correspond, for example, to a particular cardiac signal or the expiration of a timer. To illustrate, trigger could be set so ECU 100 records data from electrodes 30 before, during, and after an arrhythmia breakout or disappearance. One possible way to capture the data occurring just prior to the particular cardiac signal would be to use a data buffer that stores data (which may later be obtained) for an amount of time.

The embodiments described herein provide a catheter that may be used with the systems described above. The catheter includes a balloon and at least one optical array for mapping and/or ablating tissue, as described herein.

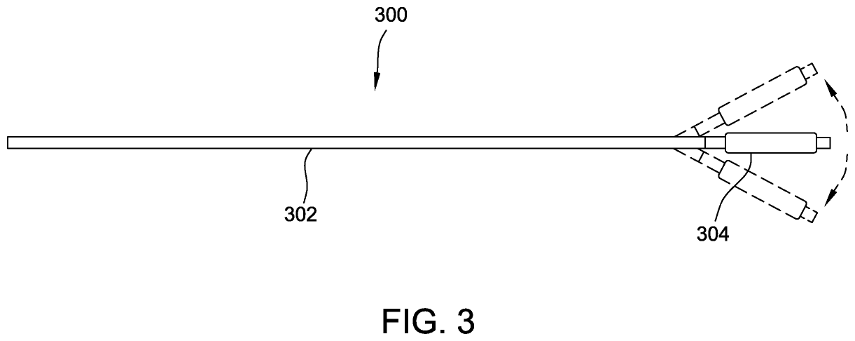

For example, FIG. 3 is a schematic side view of one exemplary embodiment of an optical balloon catheter 300. Catheter 300 may be used, for example, with system 10. Catheter 300 is deployable to a target tissue location, and is capable of performing both mapping and ablation at the target tissue location, as described herein. Catheter 300 includes a steerable shaft 302 coupled to a distal section 304. As shown in FIG. 3, distal section 304 is transitionable (e.g., rotatable) between a plurality of different positions to facilitate contacting, mapping, and ablating tissue.

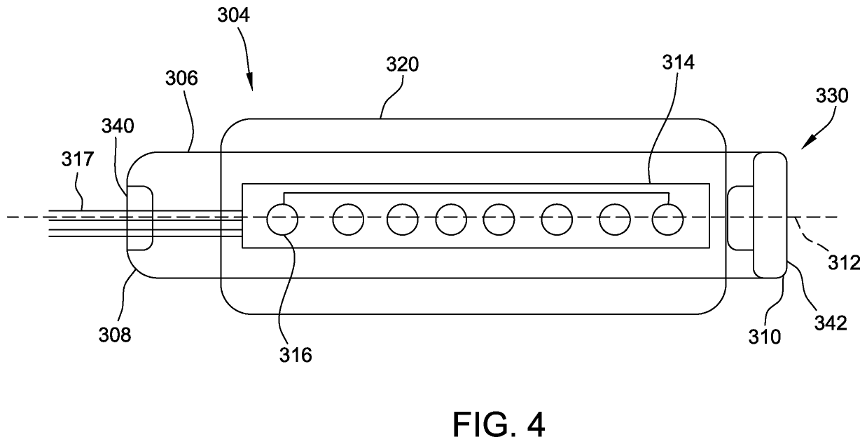

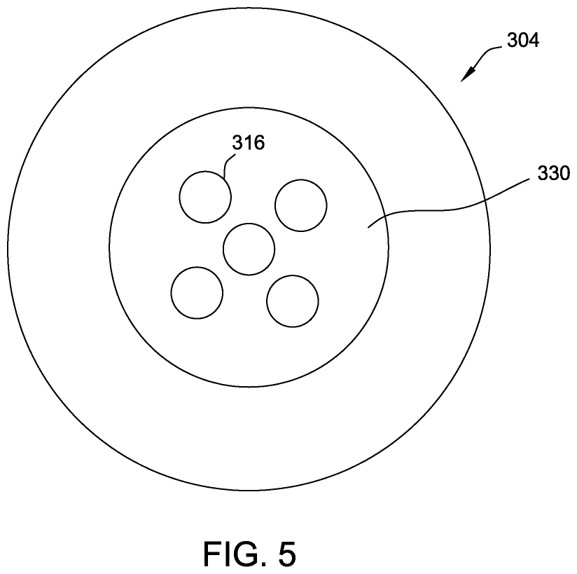

FIG. 4 is a schematic side view of distal section 304, and FIG. 5 is a schematic end view of distal section 304. Distal section 304 includes a housing 306 extending from a proximal end 308 to a distal end 310 along a longitudinal axis 312. A first optical array 314 is mounted to housing 306 between proximal end 308 and distal end 310, and extends in a direction parallel to longitudinal axis 312.

As used herein, an ‘optical array’ includes at least one optical unit. For example, an optical array may include a single optical unit moveable (e.g., via translation and/or rotation) between a plurality of different positions within the array. Alternatively, an optical array may include a plurality of optical units that have fixed positions within the array.

First optical array 314 includes at least one optical device 316. Optical devices 316 may include laser light sources, detectors, and/or transducers capable of sensing at least one tissue property (e.g., for mapping) and/or delivering ablation energy to tissue. Optical devices 316 are connected to optical fibers 317.

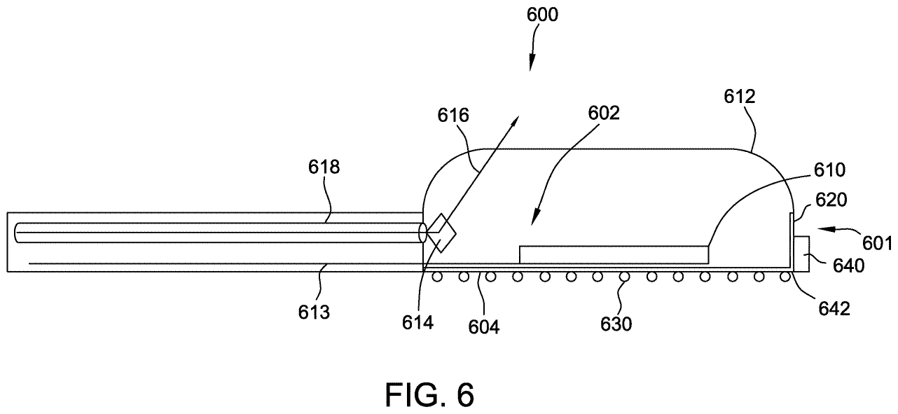

In this embodiment, a balloon 320 is coupled to housing 306, and first optical array 314 is positioned within balloon 320. That is, when catheter 300 is implanted in a subject, balloon 320 isolates first optical array 314 from the blood surrounding distal section 304, creating a clear optical pathway between optical devices 316 and tissue surfaces. Balloon 320 is generally optically transparent. However, in some embodiments, at least a portion of balloon 320 (i.e., a portion not in a field of view of optical devices 316) may be painted or otherwise coated or covered with a light-absorbing (e.g., black) material. Further, in some embodiments, a portion of balloon 320 may be covered by a light-absorbing (e.g., black) membrane, similar to what is described below in association with FIGS. 6-11. Balloon 320 is selectively inflatable, such that a user can control whether or not balloon 320 is inflated. When balloon 320 is inflated, an outer surface of balloon 320 generally contacts tissue to be mapped or ablated. Balloon 320 may be selectively inflated, for example, by pumping a fluid (e.g., water or contrast agent) into and out of balloon 320.

In this embodiment, as best shown in FIG. 5, distal section 304 further includes a second optical array 330 coupled to distal end 310 of distal section 304. Second optical array 330 also includes at least one optical device 316. Optical devices 316 in second optical array 330 are also connected to optical fibers 317. Optical devices 316 in second optical array 330 may also include laser light sources, detectors, and/or transducers capable of sensing at least one tissue property (e.g., for mapping). Optical devices 316 in second optical array 330 may be used to sense the same or different tissue properties from optical devices 316 in first optical array 314.

In this embodiment, unlike first optical array 314, second optical array 330 is not positioned within a balloon (i.e., second optical array 330 is exposed to the environment surrounding distal section 304). In some embodiments, catheter 300 includes pores (not shown) on balloon 320 and/or distal end 310 that enable flushing blood away from a tissue surface to be mapped or ablated.

To map or ablate relatively smooth surfaces, balloon 320 contacts and is swept along the tissue surface, allowing first optical array 314 to perform mapping or ablation. For mapping or ablating difficult to reach or relatively uneven surfaces (e.g., the left ventricle), second optical array 330 is used in a point by point manner.

Referring back to FIG. 4, distal section 304 further includes a first position sensor 340 at proximal end 308 of housing and a second position sensor 342 at distal end 310 of housing 306. First and second position sensors 340 and 342 facilitate determining a precise position and orientation of distal section 304. For example, first and second position sensors 340 and 342 may be electrical sensors detectable using an electric-field based system and/or magnetic sensors detectable using a magnetic field-based system, as described above.

Catheter 300 may be used for mapping and ablating both endocardial and epicardial surfaces. Further, the arrangement of first optical array 314 on the side of distal section 304 generally makes mapping and ablation much easier than in at least some known catheter systems. Specifically, first optical array 314 emits light outwards from one side of distal section 304, allowing for targeted mapping and ablation.

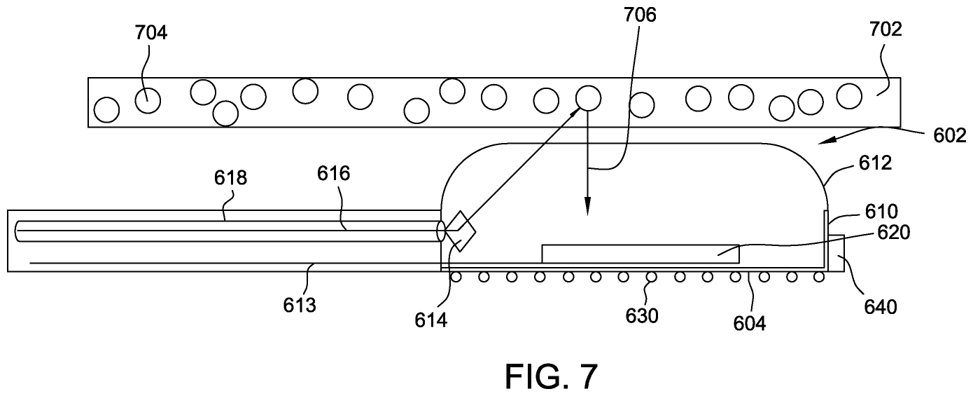

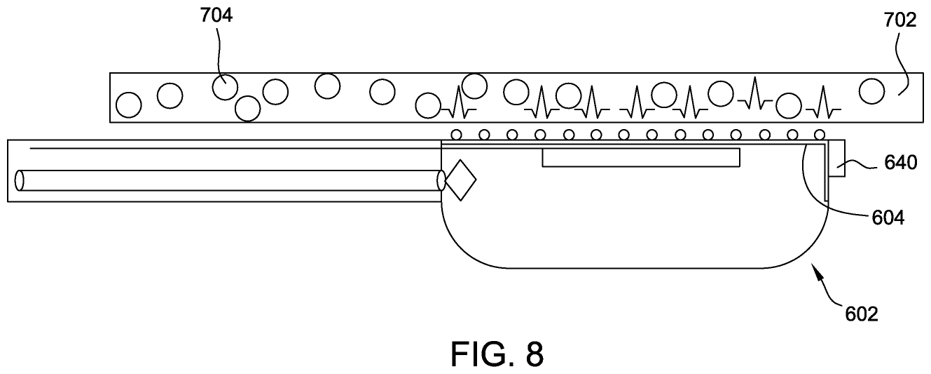

FIG. 6 is a schematic side view of an alternative balloon catheter 600. Catheter 600 includes a distal section 601 having both an optical array 602 and an electrode array 604 for mapping tissue. FIG. 7 shows optical array 602 being used to map tissue 702, and FIG. 8 shows electrode array 604 being used to map tissue 702.

In this embodiment, optical array 602 includes an optical sensor 610 (e.g., a charge-coupled device (CCD) photon detector) within a balloon 612. Optical sensor 610 is connected to a signal line 613 for communicating signals received by optical sensor 610.

Like balloon 320 (shown in FIG. 3), balloon 612 is selectively inflatable to contact tissue to be mapped. Balloon 612 may be selectively inflated, for example, by pumping a fluid (e.g., water or contrast agent) into and out of balloon 612.

Further, as in catheter 300, balloon 612 is generally optically transparent and creates an optical path and prevents blood from causing interference for optical array 602. Optical array 602 also includes a prism 614. Prism 614 receives excitation light 616 from an optical cable 618, and redirects excitation light 616 towards tissue 702. In this embodiment, tissue 702 is perfused with an imaging reagent 704. For example, imaging reagent 704 may be injected into the patient (e.g., by an intravenous or intracoronary injection) before mapping is to take place. During mapping, excitation light 616 excites the imaging reagent 704, causing photons 706 to be emitted from tissue 702. The emitted photons 706 are subsequently detected by optical sensor 610.

Different wavelengths of excitation light 616 may be transmitted towards prism 614 depending on the particular imaging reagent used. Accordingly, different imaging reagents targeting different biomarkers (e.g., innervation, inflammation, fibrosis, etc.) may be injected and sequentially activation by different wavelengths of excitation light 616.

To increase a signal to noise ratio of optical array 602, a portion of balloon 612 proximate optical sensor 610 is covered by a light-absorbing (e.g., black) membrane 620. Membrane 620 prevents extraneous light from reaching optical sensor 610 and also prevents photons 706 from reflecting off of balloon 612 and subsequently reaching optical sensor 610. Alternatively, a portion of balloon 612 may be painted or otherwise coated or covered with a light-absorbing (e.g., black) material.

Electrode array 604 includes a plurality of electrodes 630. To map tissue 702 using voltage mapping techniques, electrodes 630 are placed in contact with tissue 702, as shown in FIG. 8. Electrode array 604 may be used to sense the same tissue properties or different tissue properties than optical array 602.

In this embodiment, catheter 600 includes a position sensor 640 at a distal end 642 of catheter 600. Position sensor 640 facilitates determining a precise position and orientation of distal section 601. For example, position sensor 640 may be an electrical sensor detectable using an electric-field based system and/or a magnetic sensor detectable using a magnetic field-based system, as described above.

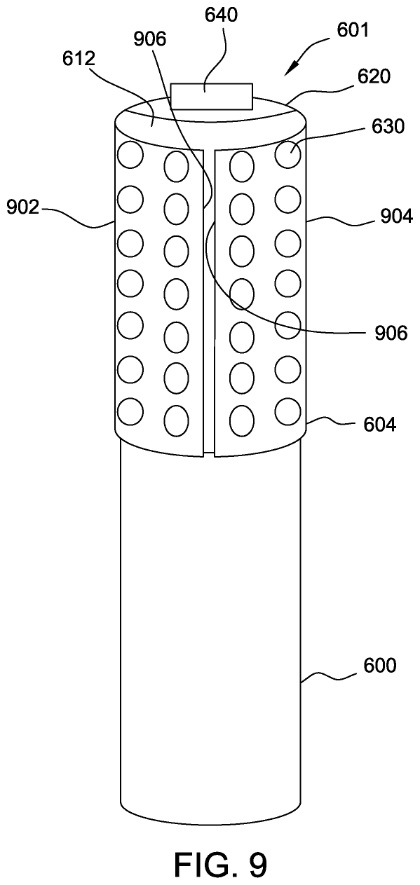

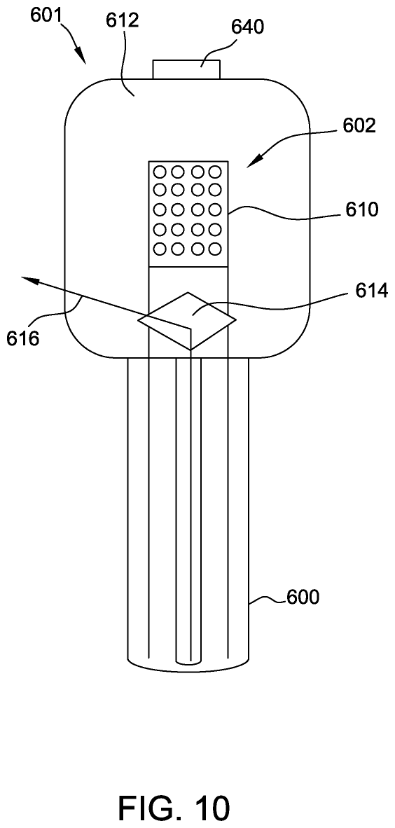

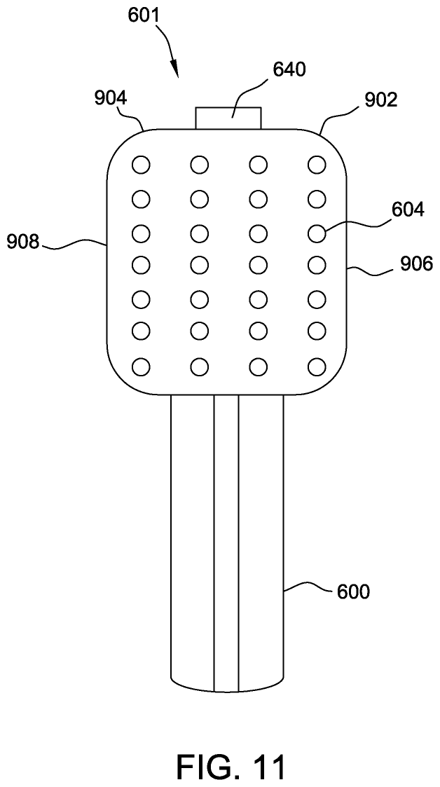

In one embodiment, inflating balloon 612 causes distal section 601 to transition between a collapsed configuration and an expanded configuration. FIG. 9 shows distal section 601 in the collapsed configuration, FIG. 10 shows optical array 602 on distal section 601 in the expanded configuration, and FIG. 11 shows electrode array 604 on distal section 601 in the expanded configuration.

Specifically, in this embodiment, electrode array 604 includes a first panel 902 and a second panel 904 that each are coupled to a plurality of electrodes 630. First panel 902 has a first edge 906 and second panel 904 has a second edge 908. First panel 902 and second panel 904 may be at least partially formed by membrane 620. As shown in FIG. 9, in the collapsed configuration, first and second panels 902 and 904 at least partially cover deflated balloon 620 and optical array 602. In addition, in the collapsed configuration, first and second edges 906 and 908 are proximate one another. Further, in this embodiment, membrane 620 is fabricated from a material having a shape memory that causes membrane 620 to at least partially envelop and contain deflated balloon 612 in the collapsed configuration. For example, membrane 620 may be a shape memory polymer.

When balloon 612 is inflated, first and second panels 902 and 904 rotate outward, exposing optical array 602 (see FIG. 10). The level of inflation of balloon 612 generally corresponds to the distance of optical array 602 from tissue 702. Further, in the expanded configuration, first and second panels 902 and 904 are positioned such that first and second edges 906 and 908 are opposite one another (see FIG. 11).

During delivery of catheter 600, distal section 601 may be in the collapsed configuration to reduce a delivery profile of catheter 600. Once catheter 600 reaches a target tissue site, distal section 601 may be transitioned to the expanded configuration to facilitate mapping tissue 702. In some embodiments, however, electrode array 604 may be used in the collapsed configuration to map tissue 702.

In the embodiments described herein, activation mapping can be accomplished optically by introducing a voltage-sensitive dye to the tissue. The voltage-sensitive dye will illuminate as an electrical activation wavefront passes through the tissue. Accordingly, the voltage-sensitive dye may be used to study normal and diseased cardiac activation patterns, including atrial and ventricular arrhythmias. The voltage-sensitive dye may have a voltage-dependent optical response time on the order of microseconds, allowing for high spatial and temporal imaging of the heart that at least some known contact electrode mapping techniques cannot provide. Further, the voltage-sensitive dye may be introduced, for example, through the coronary artery system of the subject. One example of a voltage-sensitive dye that may be used is indocyanine green (ICG).

Further, in some embodiments, calcium-sensitive dyes may be used to visualize and record calcium transients in the tissue, helping to reveal myocardial physiology and disease conditions in the heart. For example, simultaneous imaging of calcium transients and action potentials acquired using optical imaging on the epicardial surface may reveal origins of premature ventricular contraction (PVC) in subjects.

In addition, using the embodiments described herein, substrate mapping can be accomplished by leveraging differences in tissue properties. For example, for optical properties, the density, structure, and water content of tissue can significantly modify light reflection, scattering, and absorption. Optical coherence tomography, for example, is capable of leveraging these properties to detect the presence of fibrosis in cardiac tissue. Catheter 300 (shown in FIGS. 3-5), for example, may leverage similar principles to provide the same information. Further, catheter 600 (shown in FIGS. 6-11) is capable of retrieving similar information by transmitting different emission wavelengths (which may or may not be polarized) towards the tissue and, using optical sensor 610, collecting different reflection, scattering, and absorption parameters for each wavelength. Notably, optical substrate mapping does not require preparation of the tissue or biomarkers, reducing procedure time and complexity.

In other embodiments, similar to voltage- or calcium-sensitive dyes, biomarkers that are sensitive to sources or byproducts of metabolic processes may be introduced to differentiate tissue types using the optical sensing devices described herein. For example, there is a substantial difference in metabolic rates between cardiac myocytes, fibroblast cells, and scar tissue. For instance, fluorescence-labeled glucose, such as 2-NBGD, may be used to directly monitor glucose uptake by living cells and tissues.

Notably, using the embodiments described herein, both electrical signals and mechanical tissue response are detectable, and can be linked to one another, significantly improving understanding of the tissue behavior.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A catheter comprising:
a distal section comprising:
an optically transparent balloon;
a first optical array positioned within the balloon, wherein the first optical array is configured to at least one of ablate tissue and sense at least one tissue property; and
a second optical array positioned outside the balloon, wherein the second optical array is configured to at least one of ablate tissue and sense at least one tissue property.

2. The catheter of claim 1, wherein the second optical array is positioned at a distal end of the distal section.

3. The catheter of claim 1, wherein the distal section is rotatable between a plurality of different positions.

4. The catheter of claim 1, wherein the first optical array is positioned on a side of the distal section.

5. The catheter of claim 1, wherein the first optical array is oriented to emit light out one side of the distal section, and wherein the second optical array is oriented to emit light out of an end of the distal section.

6. The catheter of claim 1, further comprising a light-absorbing membrane that covers a portion of the balloon.

7. A catheter comprising:
a distal section comprising:
an optically transparent balloon;
an optical array positioned within the balloon, wherein the optical array is configured to at least one of ablate tissue and sense at least one tissue property; and
an electrode array comprising a plurality of electrodes, wherein the electrode array is configured to sense at least one tissue property.

8. The catheter of claim 7, wherein the optical array comprises:
a prism configured to redirect excitation light towards target tissue perfused with an imaging reagent; and
an optical sensor configured to sense a response of the target tissue to the excitation light.

9. The catheter of claim 7, further comprising a light-absorbing membrane that covers a portion of the balloon.

10. The catheter of claim 7, wherein, by inflating the balloon, the distal section is transitionable between a collapsed configuration and an expanded configuration.

11. The catheter of claim 10, wherein the electrode array comprises first and second panels, and wherein the first and second panels at least partially cover the balloon in the collapsed configuration.

12. A method of using a catheter, the method comprising:
deploying the catheter to a target tissue location, the catheter including a distal section having optically transparent balloon, a first optical array positioned within the balloon, and at least one of i) a second optical array positioned outside the balloon, and ii) an electrode array comprising a plurality of electrodes; and
using at least the first optical array, at least one of i) sensing at least one property of the target tissue, and ii) ablating the target tissue.

13. The method of claim 12, wherein sensing at least one property of the target tissue comprises:
perfusing the target tissue with an imaging reagent;
redirecting excitation light towards the target tissue using a prism of the first optical array; and
sensing a response of the target tissue to the excitation light using an optical sensor of the first optical array.

14. The method of claim 12, wherein sensing at least one property of the target tissue comprises:
introducing a voltage-sensitive dye to the target tissue; and
detecting, using the first optical array, illumination of the target tissue as an electrical activation wavefront passes through the target tissue.

15. The method of claim 12, wherein sensing at least one property of the target tissue comprises:
transmitting, using the first optical array, a plurality of different emission wavelengths towards the target tissue; and
sensing, using the first optical array, reflection, scattering, and absorption parameters for each wavelength.

16. The method of claim 12, wherein deploying the catheter comprises:
maneuvering the distal section to the target tissue while the balloon is deflated; and
inflating the balloon once the catheter reaches the target tissue.

17. The method of claim 16, wherein inflating the balloon causes first and second panels including the electrode array to uncover the first optical array.

18. The method of claim 12, wherein ablating tissue comprises emitting, using the first optical array, light out of one side of the distal section.

19. The method of claim 12, wherein ablating tissue comprises emitting, using the second optical array, light out of an end of the distal section.

20. The method of claim 12, further comprising determining a position and orientation of the catheter using at least one positioning sensor on the distal section.
